(19) United States
(12) Patent Application Publication
(10) Pub. No.: US 2014/0005511 A1
(43) Pub. Date: Jan. 2, 2014

(54) Implantable Medical Tube Arrangement

(71) Applicant: BIOTRONIK SE & Co. KG, Berlin (DE)

(72) Inventors: Pierre Weitzig, Berlin (DE); Gernot Kolberg, Berlin (DE)

(21) Appl. No.: 13/920,457

(22) Filed: Jun. 18, 2013

Related U.S. Application Data
(60) Provisional application No. 61/666,934, filed on Jul. 2, 2012.

(51) Int. Cl. A61N 1/05; A61B 5/0492; A61B 5/04

(52) U.S. Cl. CPC A61N 1/0551; A61B 5/04001; A61B 5/0492
USPC 600/373; 607/116

(57) ABSTRACT

An implantable medical tube arrangement including a tube wall which is resistant to bending loads, tensile loads and radial compressive loads without being damaged and is formed from at least one profiled part which is wound in a coil-shaped manner with the windings abutting against each other, wherein the profiled part's side edge running in the direction of rotation of the coil is connected to the side edge of the adjacent section of the profiled part by means of an engaging mechanism. At least one electrically conductive wire- or rope-like insertion strand is connected to the profiled par. An electrically conductive configuration of the profiled part itself is also possible.

### CROSS-REFERENCE TO RELATED APPLICATIONS

This patent application claims the benefit of co-pending U.S. Provisional Patent Application No. 61/666,934, filed on Jul. 2, 2012, which is hereby incorporated by reference in its entirety.

### TECHNICAL FIELD

The present invention relates to an implantable medical tube arrangement comprising a tube wall which is resistant to bending loads, tensile loads and radial compressive loads without being damaged.

### BACKGROUND

Such tube arrangements are primarily known and used in the field of electrode catheters; however, other fields of use are (partially) implantable tubes without electrical functionality, such as, for example, infusion tubes for external or implantable infusion pumps.

In the following, the problems underlying the present invention shall be illustrated by means of the example of an electrode catheter. Such electrode catheters have a coiled feed line formed as a metallic wire- or rope-like insertion strand which is stabilized by an electrode body made of an insulation material. This electrode body absorbs radial point or line loads and distributes the loads over a larger region of the coil. Thus, the line load is substantially converted into an area load. The stiffness of the insulation material of the electrode body reduces the bending stress of the coil when a bending load occurs. Furthermore, the electrode body insulates the coil with two different objectives. Thus, the electrode body prevents, on the one hand, a contact between the coil and penetrating body fluids which would cause corrosion on the coil. On the other hand, the coil is of course also shielded toward the body in order to prevent an electrical action on the body along the tube arrangement at places which are not intended for this. Moreover, different poles within the electrode body are electrically insulated from each other. Thus, for example, the ropes or filaments of the coil can be insulated individually or can be slid into each other through an axial tube.

For achieving the above-outlined objects, the insulation body surrounding the coil needs a tube wall having a wall thickness as great as possible. However, this results in tube arrangements with undesirable large diameters. Furthermore, a tube arrangement which is strong enough to keep the radial point and line loads away from the electrical feed line has to be very thick or stiff, which is counterproductive with regard to important product properties, such as, for example, bending properties and a small diameter.

Conventional insulation bodies have tube arrangements which are able to absorb tensile stresses only to a limited extent. For example, it has been observed that coil elongation occurs repeatedly during the extraction of an electrode catheter because the electrode construction around the electrode body is not able to absorb the required tensile stresses. Also, with regard to the occurring bending stresses in the coil, a conventional electrode body must have an adequately stiff tube arrangement in order to prevent fractures due to bending stresses.

Based on the described problems of the prior art, it is an object of the present invention to provide an implantable medical tube arrangement which, while having compact dimensions of the tube wall, has an improved load absorption behavior with regard to radial loads, bending stresses and tensile loads.

The present invention is directed toward overcoming one or more of the above-identified problems.

### SUMMARY

At least this object is achieved according to the present invention in that the tube wall is formed from at least one profiled part wound in a coil-shaped manner, wherein the profiled part's side edge running in the direction of rotation of the coil is connected to the side edge of the adjacent section of the profiled part by means of an engaging mechanism. Furthermore, the profiled part has an electric conductor or is equipped in an electrically conductive manner.

The solution according to the present invention has different advantages. For example, the bending radius of the tube arrangement is constructionally limited through the configuration of the engaging mechanism. The insertion strand which, for example, is formed by feed lines during the formation of the tube arrangement as an electrode catheter, is optimally protected against harmful bending stresses such as, for example, buckling. In addition, protection against torsion, which in the case of a coil results from a tensile load, and against squeezing is provided. The latter happens, for example, if the electrode is fixed with a ligature. Usually, a suture sleeve is used for this. If the ligature is over tightened, the conventional coil formation can be destroyed. Furthermore, the tube arrangement can absorb a high tensile force without elongating. The insertion strand itself does not absorb any forces at all. The latter is therefore optimally protected against tensile loads.

The tube arrangement is also very resistant against damages with respect to radial forces, such as, for example, radial compression which occurs in the case of a so-called subclavian crash or when applying a ligature. Also, an additional insulation of the metallic insertion strand is not required because the insertion strand is sufficiently insulated through its arrangement on or in the profiled part.

In summary, it is possible with the tube arrangement according to the present invention to use other materials and smaller dimensions for the at least one insertion strand in the tube wall because the insertion strand no longer has to absorb mechanical forces. Thus, in the case of a so-called composite wire as the feed line forming the insertion strand of a tube arrangement formed as an electrode catheter, the stabilizing MP35N layer, which in the case of conventional MP35N/silver coils absorbs any stresses, can be greatly reduced or can even be eliminated.

Advantageous refinements of the tube arrangements according to the present invention are specified in the dependent claims. In terms of the generation of the electrically conductive structure of the tube arrangement, at least one metallic wire- or rope-like insertion strand can be connected to the profiled part. Alternatively thereto, it is possible to equip the profiled part itself in an electrically conductive manner, thus to provide it with a conductive conducting layer which is applied galvanically or, for example, by using a screen printing method, or to configure it as an electrically conductive profiled part, in particular as a profiled part made of metal or as a profiled part consisting of electrically conductive plastic. In this case, this profiled part is to be electrically shielded in an advantageous manner by means of a customized insulation.

With regard to the configuration of the profiled part or parts, in particular with regard to the engaging mechanism, likewise, a multiplicity of advantageous variants is possible. For example, the profiled part can have a substantially strip-shaped cross-sectional contour, wherein on the side edges running in the longitudinal direction of the profile, in each case one engaging mechanism is arranged. In this manner, a relatively thin tube wall can be implemented.

Further preferred refinements relate to the configuration of the engaging mechanism which forms a kind of positive connection. In particular, grooves can be provided which are mutually open in the radial direction of the tube arrangement and which comprise web projections radially engaging therein with axial play. The positive-locking engagement can be intensified in that the mentioned grooves are undercut in the axial direction of the tube arrangement, and the web projections can have protrusions which, accordingly, point in the axial direction. In this configuration, the loadability of the tube arrangement in the direction of the tensile force is further improved without impairing the bendability.

The connection of the insertion strand to the profiled part can likewise be carried out in different preferred manners. For example, the insertion strand can be embedded directly into the profiled part, which preferably can be achieved by coextruding the insertion strand together with the profiled part. Alternatively thereto, the insertion strand can be inserted in each case in a receiving channel which is formed in the profiled part and is preferably open in the axial direction of the tube arrangement, and in which the insertion strand can be adhesively bonded or mechanically retained. The latter can be carried out by clipping-on or clamping. In both variants, sufficient insulation of the insertion strand is ensured. The receiving channel itself can run approximately centered with regard to the width of the profiled part or can run at one of the two side edges thereof.

According to another preferred embodiment, the profiled part can be multi-pieced consisting of an inner and an outer profiled strand which are strung together in the axial direction of the tube arrangement and are alternately engaged with each other. In this manner it is possible, for example, to stabilize an outer part guiding the insertion strand with a clamp-like inner part.

In a preferred embodiment of the tube arrangement as an electrode line, the insertion strand functions as an electrically conductive, coiled feed line to an electrical pole, sensor or actuator at a distal end of said electrode line.

The material for the profiled part can be selected from one or a plurality of the following materials: PEEK, polyurethane, polyamide, polyimide, PTFE, ETFE, silicone, polysulfone, copolymers from the aforementioned materials, high-density polyethylene, polyethylene, perfluoroethylenepropylene-copolymer (FEP), or from ceramic materials such as Al₂O₃, ZrO₂, TiO₂, MgO, ZnO, aluminum titanate (Al₂O₃+TiO₂), barium titanate (BaO+TiO₂), silicon carbide (SiC), beryllium oxide (BeO), aluminum nitride (AlN), Hafnium carbide (HfC), tantalum carbide (TaC), titanium nitride (TiN), boron nitride (BN), boron carbide (B₄C), tungsten carbide (WC), silicon nitride (Si3N4), metal, electrically conductive plastic, glass, and the like.

When using one of the mentioned ceramic materials, which usually can be sintered, it is of advantage for production-related reasons to wind the profiled part prior to sintering into a coil and to subsequently carry out the sintering process. In the case of an embedded insertion strand, the latter is coextruded together with the ceramic material and subsequently firmly embedded through the sintering process. In the case of, for example, a clipped-in insertion strand, this joining step can be carried out after sintering the profiled part.

For the material of the insertion strand, preferably, a normal rope made of a medical, conductive stranded wire, or a coiled wire or a profile made of a conductive plastic is conceivable.

The structure of the engaging mechanism, thus, for example, the kind of positive connection of the engaging components of the profiled parts can be varied over the length of the tube arrangement so that said tube arrangement has variable bending properties at different axial positions. In this manner, different bending radii can be implemented at different longitudinal positions of the tube arrangement.

Further features, aspects, objects, advantages, and possible applications of the present invention will become apparent from a study of the exemplary embodiments and examples described below, in combination with the figures, and the appended claims.

### DESCRIPTION OF DRAWINGS

Further features, details and advantages of the present invention arise from the following description in which different exemplary embodiments are illustrated in more detail by means of the attached drawings. In the figures:

FIGS. 1-2 show perspective, partially cut-away views of a tube arrangement in a first embodiment.

FIG. 3 shows a perspective partial illustration of the profiled part with an electrical feed line.

FIG. 4 shows a perspective, partially cut-away view of a tube arrangement in a second embodiment.

FIG. 5 shows a perspective partial illustration of the profiled part according to FIG. 4 with two feed lines inserted therein.

FIGS. 6-8 show perspective, partially cut-away illustrations of a tube arrangement in a third embodiment.

FIG. 9 shows a perspective partial view of the profiled part according to FIGS. 6-8 with a feed line embedded therein.

FIGS. 10-12 show perspective, partially cut-away views of a tube arrangement in a fourth embodiment.

FIG. 13 shows a perspective partial view of the profiled part according to FIGS. 10-12 with four embedded feed lines.

FIGS. 14-15 show perspective, partially cut-away illustrations of a tube arrangement in a fifth embodiment.

FIG. 16 shows a perspective partial illustration of the profiled part according to FIGS. 14-15 with an embedded feed line.

FIG. 17 shows a perspective, partially cut-away illustration of a tube arrangement in a sixth embodiment.

FIG. 18 shows a perspective partial view of a profiled part according to FIG. 17 composed of two components.

FIG. 19 shows a perspective, partially cut-away illustration of a tube arrangement in a seventh embodiment.

FIG. 20 shows a perspective partial view of a profiled part according to FIG. 19 composed of two components.

FIG. 21 shows a perspective, partially cut-away illustration of a tube arrangement in an eight embodiment with four inserted feed lines.

FIG. 22 shows a perspective, partially cut-away illustration of a tube arrangement in a ninth embodiment.

FIG. 23 shows a perspective partial illustration of the profiled part used in FIG. 22.

### DETAILED DESCRIPTION

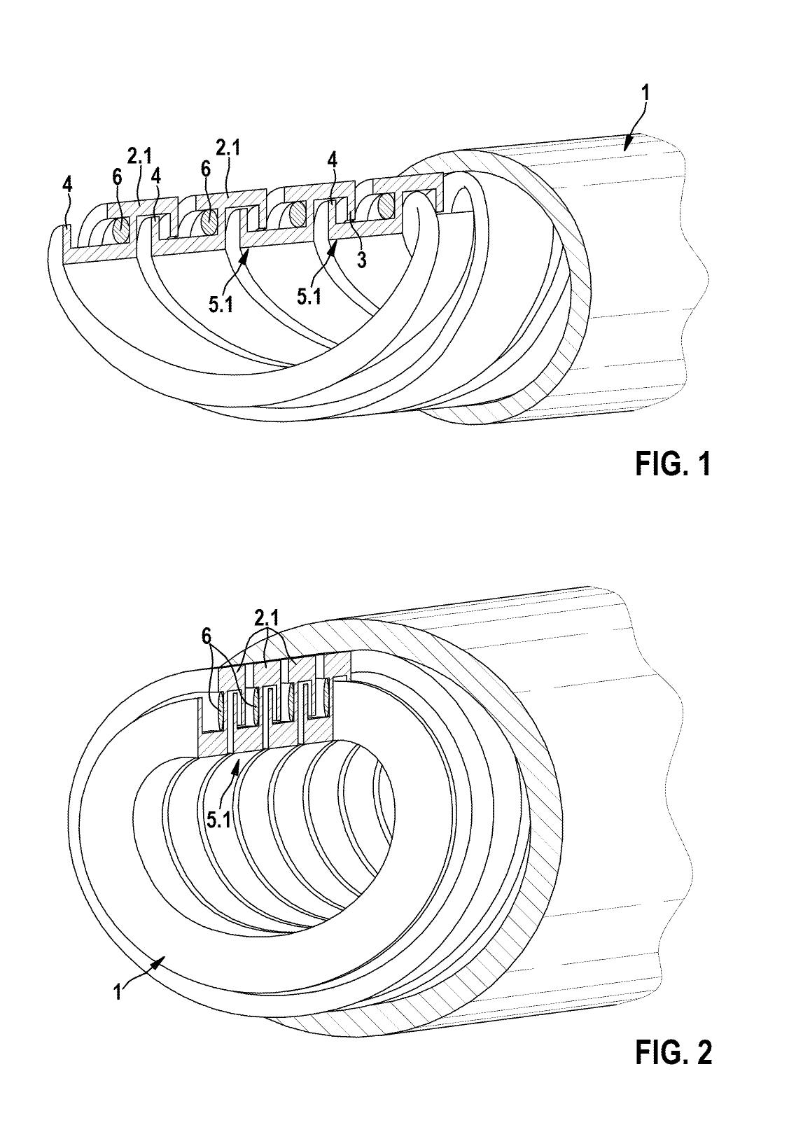

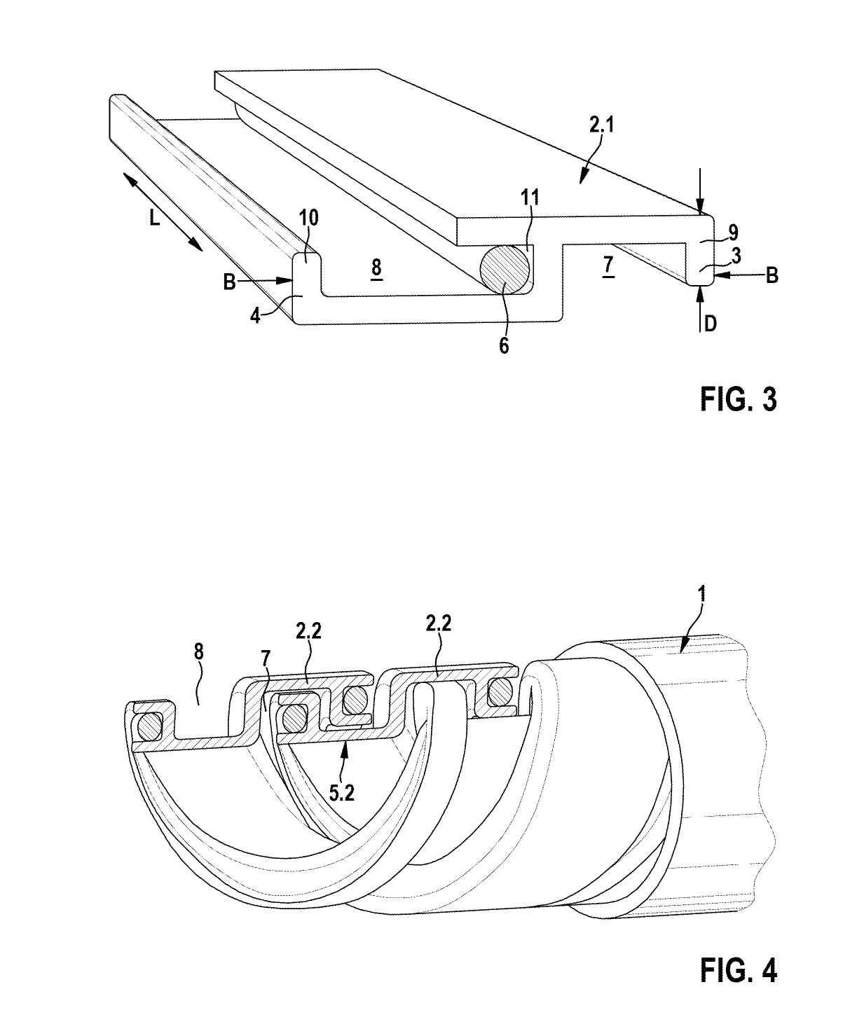

All exemplary embodiments of the shown tube arrangements are based on a consistent construction principle which shall be explained with reference to FIGS. 1-3. Thus, for structuring an elongated tube arrangement with a tube wall 1, a profiled part 2.1 is used which—as clearly shown in FIG. 3—has a strip-shaped contour. This means that the width dimension B transverse to the longitudinal direction L of the profile is many times higher than the profiled part's 2.1 thickness D forming the tube wall 1. As is apparent from FIGS. 1-2, the profiled part 2.1 is wound in a coil-shaped manner, preferably with the windings abutting against each other, so that a closed tube wall 1 is formed. In order to hold the windings of the profiled part 2.1 together, the profiled part is provided on its two side edges 3, 4 with an engaging mechanism 5.1 by means of which adjacent sections of the individual windings of the profiled part 2.1 are connected in a manner yet to be explained in more detail.

Connected to the profiled part 2.1 is a metallic insertion strand in the form of a coiled wire 6 which can be provided for electrically contacting, for example, an electrode of a cardiological electrode catheter which is not illustrated herein.

In the embodiment of the profiled part 2.1 shown in FIGS. 1-3, the engaging mechanism 5.1 is formed on one side by a groove 7, 8 which is open in the radial direction R along the two side edges 3, 4 which are bordered on the outside by web projections 9, 10 extending on the outside along the side edges 3, 4. As is particularly apparent from FIG. 1, the respective web projections 9 and 10 of the two adjacent sections of the profiled part 2.1 engage in each case with axial play in the grooves 7, 8 so that the integrity of the tube wall 1 is ensured over the length of the tube arrangement, but, at the same time, flexible bending of the tube wall 1 is enabled by displacing the respective web projections 9, 10 in the grooves 7, 8.

The coiled wire 6 of the profiled part 2.1 is clipped, glued, mechanically clamped, or otherwise secured in a centrally running receiving channel 11 which is open toward the side.

In terms of the production process, the profiled part 2.1 which consists, for example, of PEEK material, is first extruded and, then, the coiled wire 6 is inserted into the receiving channel 11. Subsequently, the four profiled parts 2.1 in the shown example are put together and wound so that a quadruple strand of wired coils 6 is fed along the tube wall 1.

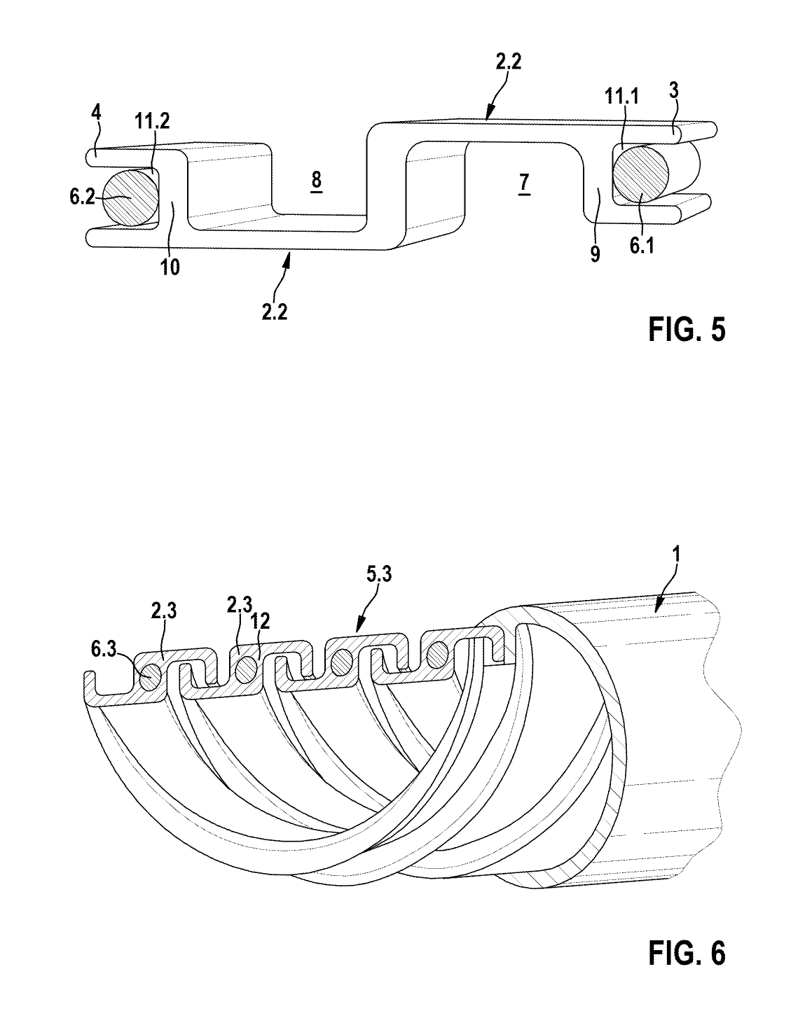

The embodiment shown in FIGS. 4-5 is based on a profiled part 2.2 in which, again, an engaging mechanism 5.2 with grooves 7, 8 and web projections 9, 10 are provided. Here, in contrast to the exemplary embodiment according to FIGS. 1-3, in one profiled part 2.2, two coiled wires 6.1, 6.2 are provided, the receiving channels 11.1, 11.2 of which are, in each case, provided along the side edges 3, 4. Here too, securing the coiled wires 6.1, 6.2 in the receiving channels 11.1, 11.2 is carried out via, for example, clipping-in, adhesive bonding, mechanical clamping, etc.

As is clearly shown in FIG. 4, the web projections 9, 10 with the receiving channels 11.1, 11.2 formed thereon lie in the respective grooves 7, 8 so that a mutual engagement with a correspondingly stable connection between the individual windings of the profiled part 2.2 takes place.

The manner of preparation takes place analogous to the exemplary embodiment according to FIGS. 1-3, wherein again a plurality of profiled parts 2.2 can be wound side by side into a multi-strand.

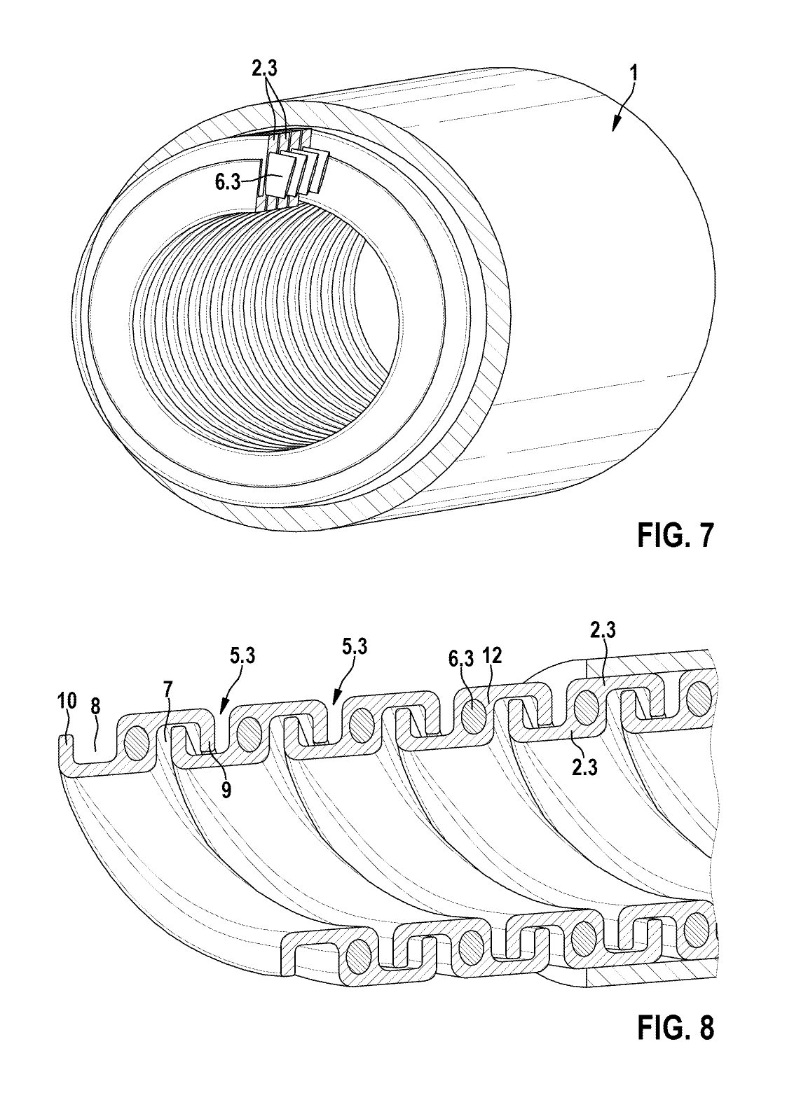

The exemplary embodiment of a tube arrangement shown in FIGS. 6-9 is based on a profiled part 2.3 which, analogous to the previous exemplary embodiments, has an engaging mechanism 5.3 with grooves 7, 8 and web projections 9, 10. Insofar, there is no significant difference to the previous exemplary embodiments with regard to the winding and the engaging connection between the windings of the profiled parts 2.3.

Deviating therefrom is the accommodation of the coiled wire 6.3 which is accommodated in FIGS. 6-9 completely embedded in a central core section 12 of the profiled part 2.3. For this purpose, the coiled wire 6.3 is coextruded with the profiled part 2.3. Subsequently, a plurality of profiled parts 2.3 are strung together again through the engaging mechanism 5.3 shown in FIGS. 6-8, and are preferably wound abutting against each other so that a continuous tube wall 1 with completely embedded coiled wires 6.3 is implemented. As is not in illustrated in more detail in these drawings, when using a plurality of profiled parts 2.3 wound on each other, it is possible to arrange blind profiled parts between the profiled parts 2.3 comprising the coiled wires 6.3.

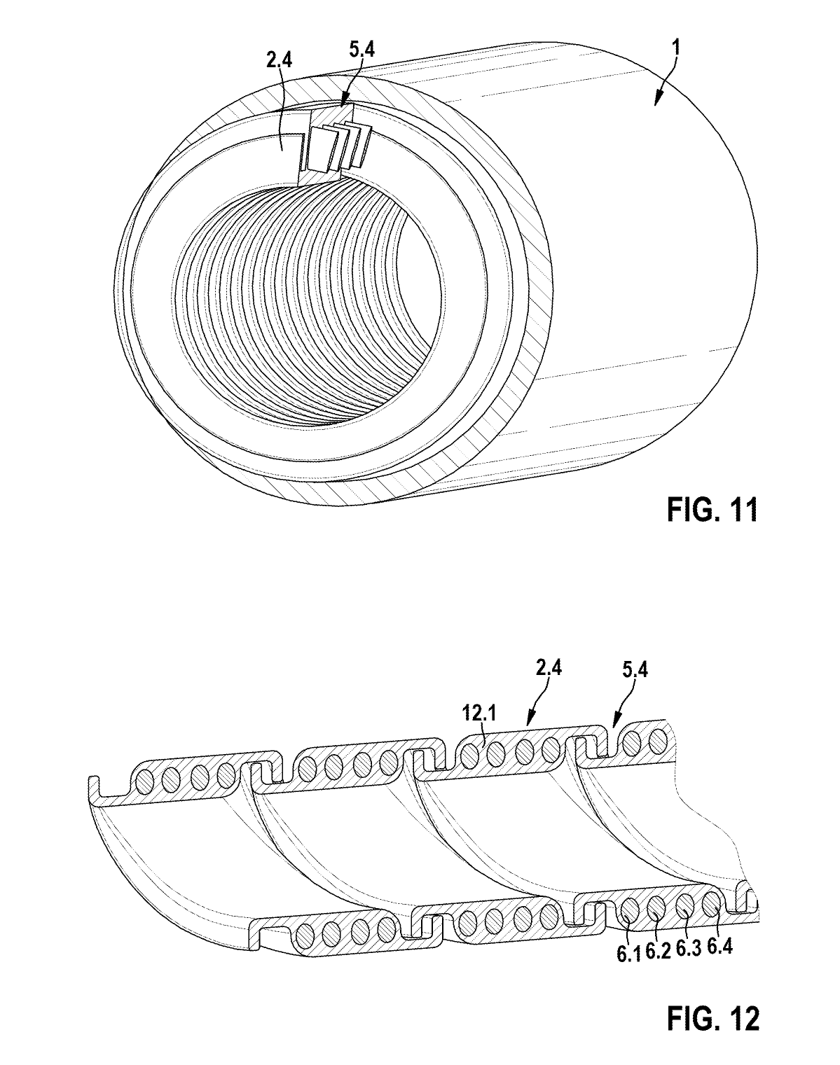

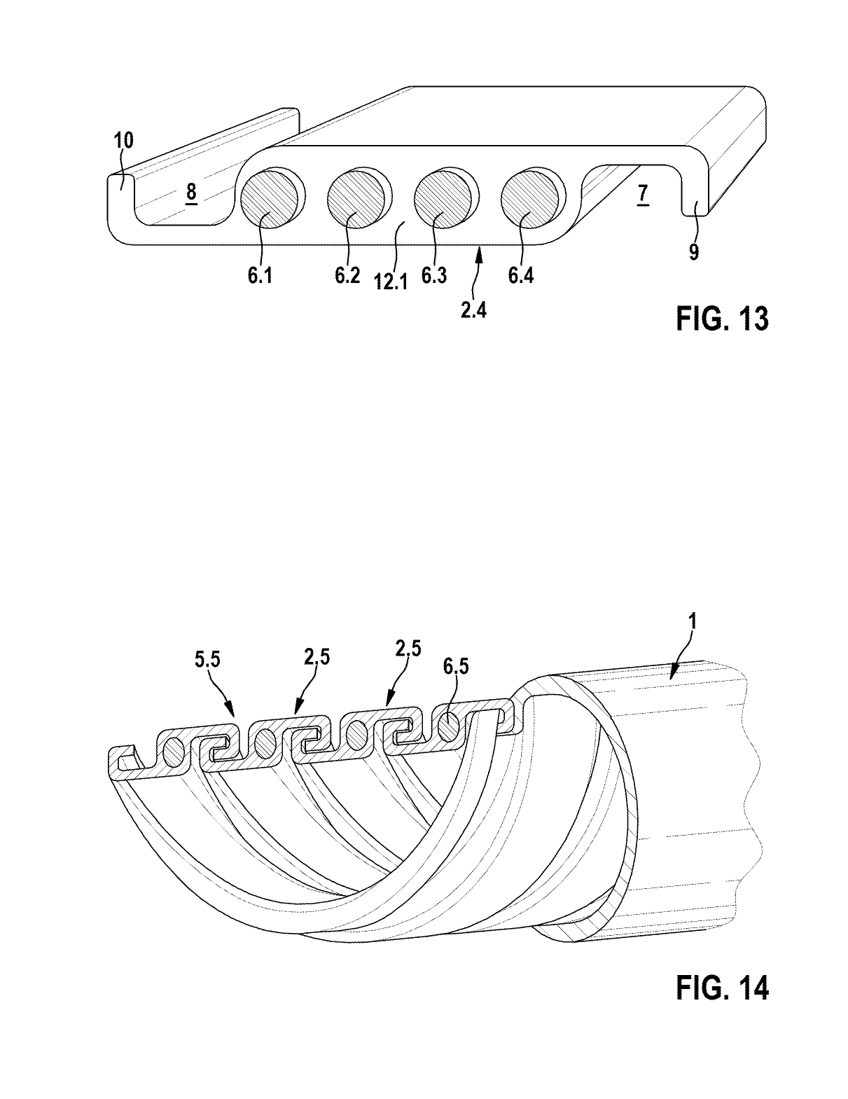

In the embodiment shown in the FIGS. 10-13, four coiled wires 6.4 are extruded parallel and next to each other into a widened core section 12.1. Subsequently, this profiled part 2.4 is preferably wound with the windings abutting against each other and is fixed with the same engaging mechanism 5.4 as it is used in the exemplary embodiments according to FIGS. 1-3 and 6-9. Thus, the web projections 9, 10 engage mutually in the grooves 7, 8 in order to ensure a tension-resistant but bendable connection between the individual windings of the profiled parts 2.4.

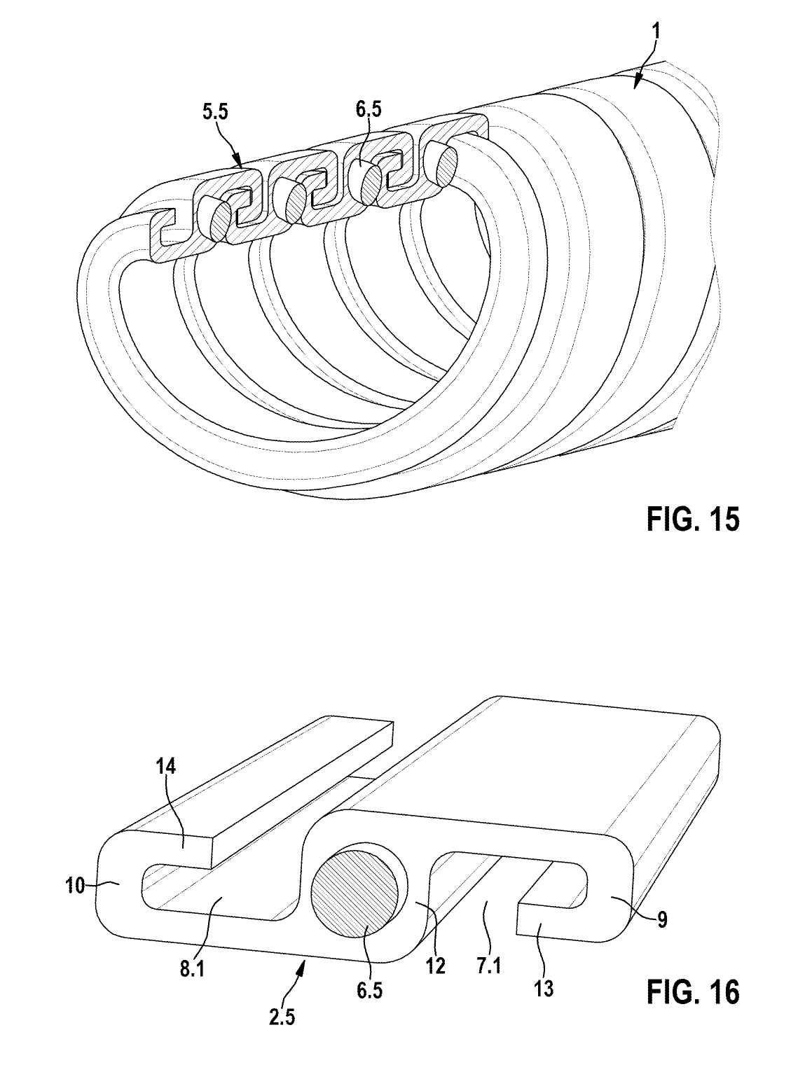

The embodiment shown in FIGS. 14-16 derives from the embodiment according to FIGS. 6-9, i.e., in that the coiled wire 6.5 again is centrally embedded in the profiled part 2.5. In contrast to the aforementioned embodiment according to FIGS. 6-9, the engaging mechanism 5.5 is additionally reinforced in that the grooves 7.1, 8.1 are undercut in the axial direction of the tube arrangement. This is implemented through protrusions 13, 14 on the web projections 9, 10, which protrusions, accordingly, point in the axial direction so that—as clearly shown in FIG. 14—here again, the adjacent windings of the quadruple profiled part 2.5 are meshed with each other. This represents a particularly stable but, at the same time, also a bendable connection. The coiled wire 6.5 is extruded into the core section 12.

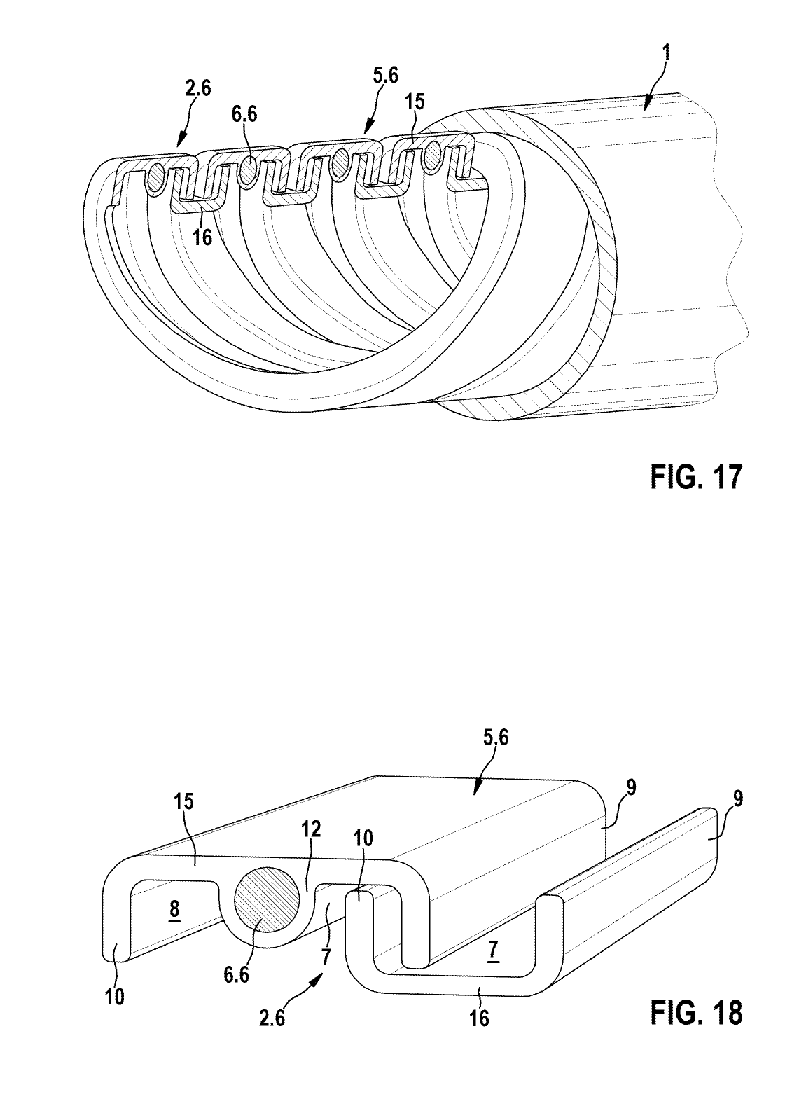

FIGS. 17-18 show a tube arrangement in which the profiled part 2.6 is composed of two components, namely, an outer profiled strand 15 and an inner profiled strand 16. Both profiled strands 15, 16 have an approximately flat U-shaped cross-section. The U-legs thus form the web projections 9, 10 of the engaging mechanism 5.6 of the profiled part 2.6, which web projections 9, 10 mutually engage in the inner space surrounded by the U-legs and the U-base of the profiled strands 15, 16 and formed as grooves 7, 8. Thus, outer and inner profiled strands 15, 16 can mutually engage with each other and can preferably be wound with the windings “abutting against each other”. The outer profiled strand 15 again carries in a core section 12 a coiled wire 6.6 which is extruded therein at the same time.

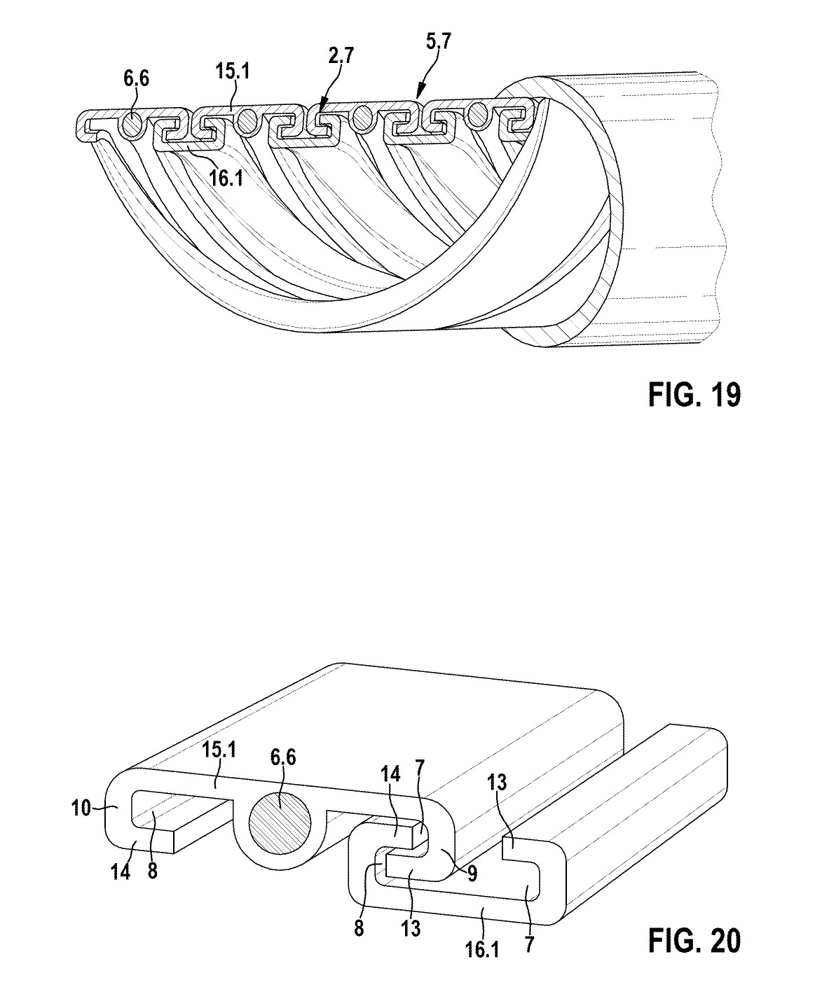

The embodiment according to FIGS. 19-20 differs from the one according to FIGS. 17-18 in that the two profiled strands 15.1, 16.1 with the undercut grooves 7, 8 through the clamp-like inwardly extending protrusions 13, 14 have an engaging mechanism 5.7 which is undercut at the webs 9, 10, as already explained in more detail by with respect to FIGS. 14-16.

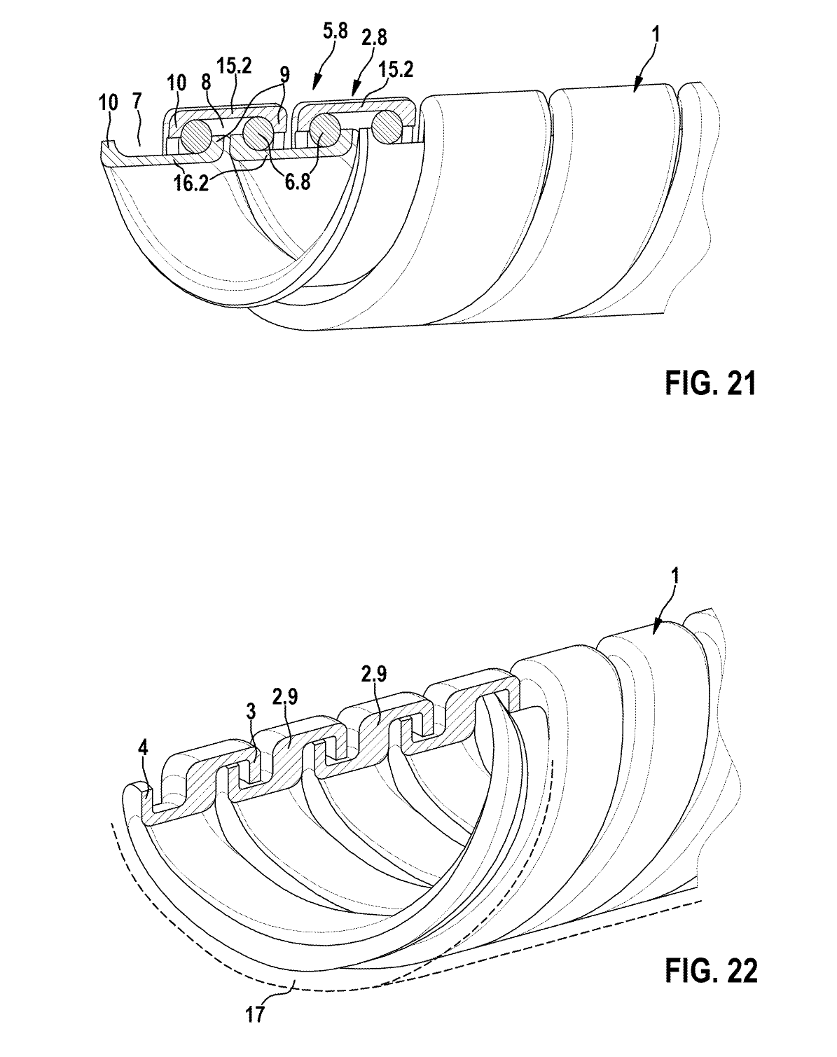

In the embodiment shown in FIG. 21, the profiled part 2.8 is formed again by an outer profiled strand 15.2 and an inner profiled strand 16.2 which are alternately offset to each other and engage with their web projections 9, 10 in the corresponding groove 7, 8 for forming the engaging mechanism 5.8. The particular feature of this embodiment is the fact that the coiled wires 6.8 are part of the engaging mechanism 5.8 in that the web projections 9, 10 also engage over the respective coiled wires 6.8.

In terms of the production process, the coiled wires are first wound with a pitch that is greater than the windings of the profiled part 2.8 and are then slid underneath the outer profiled strand 15.2 and over the inner profiled strand 16.2. Subsequently, the profiled strands 15.2, 16.2 are connected to each other so that the configuration shown in FIG. 21 is established. In this configuration, the individual profiled strands 15.2, 16.2 are held together in the axial direction, are flexibly bendable, and the coiled wires 6.8 are reliably insulated with respect to each other.

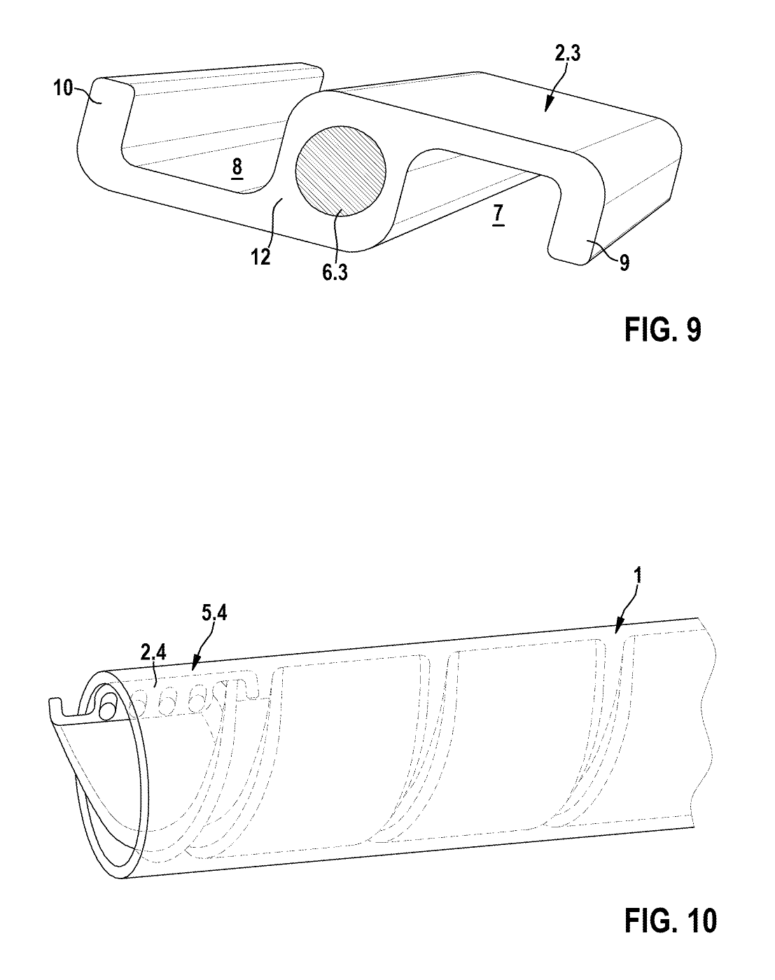

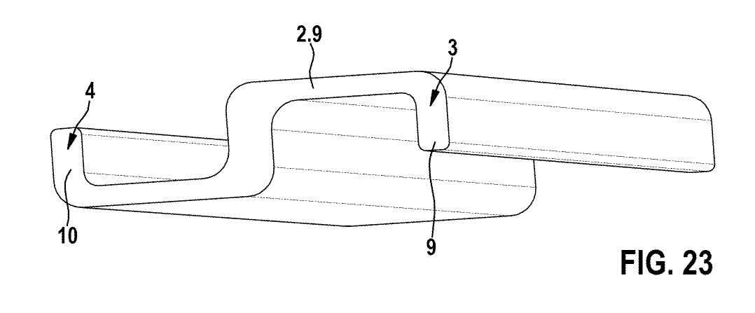

In the embodiment shown in the FIGS. 22-23, instead of the profiled part being made of plastic, a profiled part 2.9 made of metal is used, which is refined based on the usual flat strip coils and, accordingly, has the contour according to the present invention and the mutual engagement of the profiled parts analogous to, for example, the embodiment shown in FIGS. 8-9. Around the tube wall 1 formed from the profiled part 2.9, an insulation 17 is placed which is only indicated with a dashed line. The resulting coil serves as an inner or outer coil and stabilizes the electrode. The profiled part 2.9 can also entirely consist of electrically conductive plastic.

It will be apparent to those skilled in the art that numerous modifications and variations of the described examples and embodiments are possible in light of the above teachings of the disclosure. The disclosed examples and embodiments are presented for purposes of illustration only and are not meant to be limiting. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range.

I/we claim:

1. An implantable medical tube arrangement, comprising:
a tube wall which is resistant to bending loads, tensile loads and radial compressive loads without being damaged,
wherein the tube wall is formed from at least one profiled part which is wound in a coil-shaped manner, wherein a side edge of the profiled part running in a direction of rotation of the coil is connected to a side edge of an adjacent section of the profiled part by means of an engaging mechanism, and
wherein the at least one profiled part has an electrical conductor or is equipped to be electrically conductive.

2. The tube arrangement according to claim 1, wherein the profiled part is wound such that the windings abut against each other.

3. The tube arrangement according to claim 1, wherein at least one metallic wire- or rope-like insertion strand is connected to the profiled part.

4. The tube arrangement according to claim 1, wherein the profiled part is configured as an electrically conductive profiled part made of metal, or as a profiled part consisting of an electrically conductive plastic.

5. The tube arrangement according to claim 4, wherein the profiled part made of metal, or the profiled part consisting of electrically conductive plastic, is provided with insulation.

6. The tube arrangement according to claim 1, wherein when using a plurality of profiled parts wound on each other, blind profiled parts are arranged between the profiled parts which are equipped to be electrically conductive or comprise an electrical conductor.

7. The tube arrangement according to claim 1, wherein the profiled part has a strip-shaped contour, wherein at side edges of the contour running in a longitudinal direction of the profile in each case one engaging mechanism is arranged.

8. The tube arrangement according to claim 1, wherein the engaging mechanism is formed by grooves which are mutually open in a radial direction of the tube arrangement and comprise web projections radially engaging therein with axial play.

9. The tube arrangement according to claim 8, wherein the grooves are undercut in an axial direction of the tube arrangement and the web projections have protrusions which point in the axial direction.

10. The tube arrangement according to claim 3, wherein the at least one insertion strand is embedded in the profiled part by means of coextrusion.

11. The tube arrangement according to claim 3, wherein the at least one insertion strand is inserted, in each case, in a receiving channel which is formed in the profiled part and is open in the axial direction of the tube arrangement.

12. The tube arrangement according to claim 11, wherein the insertion strand is glued, or mechanically retained by clipping or clamping, in the receiving channel.

13. The tube arrangement according to claim 11, wherein the receiving channel runs approximately central with regard to a width of the profiled part.

14. The tube arrangement according to claim 11, wherein the receiving channel runs at one, or both, side edges of the profiled part.

15. The tube arrangement according to claim 1, wherein the profiled part include an inner and an outer profiled strand which are strung together in an axial direction of the tube arrangement and are alternately engaged with each other.

16. The tube arrangement according to claim 3, in the form of an electrode line for stimulating and/or sensing muscle and/or nerve activities, and/or for transmitting data or activating sensors or actuators, wherein the insertion strand is configured as an electrically conductive coiled feed line to an electrical pole, sensor or actuator at the distal end of the electrode line.

17. The tube arrangement according to claim 1, wherein material for the profiled part is selected from one of the following materials: PEEK, polyurethane, polyamide, polyimide, PTFE, ETFE, silicone, polysulfone, copolymers from the aforementioned materials, high-density polyethylene, polyethylene, perfluoroethylenepropylene-copolymer (FEP), or from ceramic materials such as Al₂O₃, ZrO₂, TiO₂, MgO, ZnO, aluminum titanate (Al₂O₃+TiO₂), barium titanate (BaO+TiO₂), silicon carbide (SiC), beryllium oxide (BeO), aluminum nitride (AlN), Hafnium carbide (HfC), tantalum carbide (TaC), titanium nitride (TiN), boron nitride (BN), boron carbide (B₄C), tungsten carbide (WC), silicon nitride (Si3N4), metal, electrically conductive plastic, or glass.

18. The tube arrangement according to claim 1, wherein when using a ceramic sinterable material for the profiled part, said profiled part is wound into a coil prior to sintering.

19. The tube arrangement according to claim 3, wherein the insertion strand consists of a rope or a coiled wire.

20. The tube arrangement according to claim 1, wherein the engaging mechanism varies over a length of the tube arrangement in such a manner that the tube arrangement has variable bending properties at different axial positions.
